(19) United States
(12) Patent Application Publication
(10) Pub. No.: US 2006/0014680 A1
(43) Pub. Date: Jan. 19, 2006

(54) Peptides and compounds that bind to the IL-5 receptor

(76) Inventors: Caiding Xu, Cupertino, CA (US); Lydie Yang, Santa Clara, CA (US); Ashok Bhandari, Milpitas, CA (US); Chris Holmes, Saratoga, CA (US); Min-Jia Chen, San Francisco, CA (US); Peter Schatz, Cupertino, CA (US); Palani Balu, Cupertino, CA (US)

Correspondence Address: BIO TECHNOLOGY LAW GROUP, 658 MARSOLAN AVE., SOLANA BEACH, CA, 92075, US

(21) Appl. No.: 10/890,546

(22) Filed: Jul. 13, 2004

(51) Int. Cl. A61K 38/16; A61K 38/08; A61K 38/10; C07K 7/64; C07K 7/08

(52) U.S. Cl. 514/12; 514/13; 514/14; 514/15; 530/324; 530/325; 530/326; 530/327; 530/328

(57) ABSTRACT

New IL-5 receptor antagonists and methods of use are described, e.g., in the treatment of IL-5 receptor mediated disorders. The compounds include both monomers and dimers that were identified using one or more of alanine scans, lysine scans, other residue substitutions, and C- and N-terminal truncations and additions vis a vis the core sequence Val Asp Glu Cys Trp Arg Ile Ile Ala Ser His Thr Trp Phe Cys Ala Glu Glu-AF17121 (SEQ ID NO: 1) and shorter sequences and derivatives thereof.

### RELATED PATENTS AND APPLICATIONS

This application is related but does not claim priority to U.S. patent application Ser. No. 08/485,302 (U.S. Pat. No. 5,668,110), U.S. patent application Ser. No. 08/478,312 (U.S. Pat. No. 5,654,276), U.S. patent application Ser. No. 08/484,083 (U.S. Pat. No. 5,683,983), U.S. patent application Ser. No. 08/476,169 (U.S. Pat. No. 5,677,280), pending U.S. patent application Ser. Nos. 09/445,582 and 10/168,372, and PCT applications PCT/GB97/01620 (WO9857991), PCT/GB97/01618 (WO9857980), PCT/EP98/03505 (WO9857979), and PCT/US99/30081 (WO01/43527). Each of the foregoing is entitled PEPTIDES AND COMPOUNDS THAT BIND TO THE IL-5 RECEPTOR and each is herein incorporated by reference in its entirety including all drawings and sequence listings.

### BACKGROUND ART

The field of the invention relates to immune system function and modulation, more particularly lymphokines, and more particularly still interleukin-5 (IL-5 or IL5), its receptor, and modulation thereof for the diagnosis, treatment, and/or prevention of disease.

IL-5 is a lymphokine secreted by T cells and mast cells, having biological activities on B cells and eosinophils. In murine hematopoiesis, IL-5 is a selective signal for the proliferation and differentiation of the eosinophilic lineage. See Yamaguchi et al., J. Exp. Med. 167: 43-56 (1988). In this respect, IL-5 function shows analogies with colony-stimulating factors for other myeloid lineages. Also, human (h) IL-5 is very potent in the activation of human eosinophils. See Lopez et al., J. Exp. Med. 167: 219-224 (1988); Saito et al., Proc. Natl. Acad. Sci. USA 85:2288-2292 (1988).

IL-5 mediates its activity through a cell membrane receptor-complex. This complex has been characterized physicochemically in both murine and human systems. Mouse pre-B cell lines depending on IL-5 for their growth have been developed from bone marrow and are used for IL-5 receptor analysis. See Rolink et al., J. Exp. Med. 169: 1693-1701 (1989). The human IL-5 receptor has been studied on a subclone of the promyelocytic cell line HL60 induced towards eosinophil differentiation. See Plaetinck et al., J. Exp. Med. 172: 683-691 (1990).

Eosinophilic differentiation is initiated using sodium butyrate. Only high affinity (Kd=30 pM) IL-5 binding sites can be found on these cells. However, cross-linking studies reveal the presence of two polypeptide chains involved in IL-5 binding, IL-5R-α and IL-5β chains.

Devos et al., Canadian Patent Publication 2,058,003 describes a recombinant chain of human IL-5R or parts thereof, DNA-sequences coding for such a receptor or parts thereof, and host cells transformed with such vectors.

Takatsu et al., European Patent Publication 475,746 provides an isolated cDNA sequence coding for murine and human IL-5 receptor. The extracellular domain (ECD) of the human IL-5R-α chain can be expressed in cells, such as CHO cells, in a manner that allows for the enzymatic harvest of the receptor from the cell surface and its subsequent immobilization using a capture antibody. See E. A. Whitehorn, et al., BioTechnology 13:1215 (1995).

A soluble human IL-5R-α chain can be used as an IL-5 antagonist in chronic asthma or other disease states with demonstrated eosinophilia. Eosinophils are white blood cells of the granulocytic lineage. Their normal function appears to be combating parasitic infections, particularly helminthis infections. However, their accumulation in tissues, a condition referred to as eosinophilia, is also associated with several disease states, most notably asthma. It is believed that the damage to the epithelial lining of the bronchial passages in severe asthmatic attacks is largely caused by the compounds released by degranulating eosinophils.

In U.S. Pat. No. 5,096,704, there is reported the use of compounds which block the stimulatory effects of IL-5 in order to inhibit production and accumulation of eosinophils. The stimulatory effects of IL-5 were blocked by administering an effective amount of an antagonist to human interleukin-5, preferably using monoclonal antibodies or binding compositions derived therefrom by standard techniques. Monoclonal antibodies were selected by their ability to inhibit IL-5 induced effects in standard IL-5 bioassays, such as the ability to stimulate the growth and development of eosinophils in in vitro colony forming assays, and the ability to augment in vitro proliferation of the in vivo passaged BCL 1 lymphoma cells. The use of antibody fragments, e. g., Fab fragments, was also reported.

Commonly-owned U.S. Pat. Nos. 5,668,110; 5,677,280; 5,654,276, and 5,683,983, and pending U.S. patent application Ser. No. 09/445,582 and 10/168,372 also discuss peptides that bind IL-5 receptors and block the effect of IL-5.

Currently glucocorticoid steroids are the most effective drugs for treating the acute effects of allergic diseases, such as asthma. However, the availability of alternative or complementary approaches to the treatment of disorders associated with eosinophilia would have important clinical utility.

The availability of cloned genes for IL-5R, including a soluble IL-5R derivative, facilitates the search for agonists and antagonists of these important receptors. The availability of the recombinant receptor protein allows the study of receptor-ligand interaction in a variety of random and semi-random peptide diversity generation systems. These systems include the “peptides on plasmids” system described in U.S. Pat. No. 5,270,170, the “peptides on phage” system described in U.S. patent application Ser. No. 718,577, filed Jun. 20, 1991, and in Cwirla et al., (1990) Proc. Natl. Acad. Sci. USA 87:6378-6382, and the “very large scale immobilized polymer synthesis” system described in U.S. Pat. No. 5,143,854; PCT patent publication No. 90/15070, published Dec. 13, 1990; U.S. patent application Ser. No. 624,120, filed Dec. 6, 1990; Fodor et al., 15 Feb. 1991, Science 251:767-773; Dower and Fodor (1991) Ann. Rep. Med. Chem. 26:271-180; and U.S. patent application Ser. No. 805,727, filed Dec. 6, 1991. Each of the foregoing patent applications and publications is incorporated herein by reference.

Asthma has become the most common chronic disease in industrialized countries. Conventional methods and therapeutic agents may not be completely effective in the treatment of asthma or other immunomediated inflammatory diseases in all patient populations. Moreover, there remains a need for compounds that bind to or otherwise interact with the IL-5R, both for studies of the important biological activities mediated by this receptor and for treatment of disease.

### SUMMARY OF THE INVENTION

The present invention provides surprisingly potent derivatives of the following IL-5 receptor ligand reported in commonly-owned application 10/168,372, wherein the order left to right is N-terminus to C-terminus: Ac-Val Asp Glu Cys Trp Arg Ile Ile Ala Ser His Thr Trp Phe Cys Ala Glu Glu-NH₂ (AF17121; SEQ ID NO:1). The peptides and peptide mimetics of the invention are preferably twelve to forty or more amino acid residues in length, preferably twelve to twenty-five amino acid residues in length, and comprise the core sequence of amino acids as shown.

In one embodiment, the invention comprises new derivatives of SEQ ID NO: 1, preferably 12 to 40 or more amino acids in length, more preferably 12 to 25 amino acid residues in length, and wherein one or more of the following residues in the core sequence is substituted with Ala: Trp at position 13, Ser at position 10, Glu at position 3, Asp at position 2, and Val at position 1. The N-terminus can be acylated or else left as a free amino group. The C-terminus may or may not be amidated; if not, a —COOH group is left at that position. Single substitution of alanine at position 12 makes the peptide monomer about 135% more potent, at position 10 about 450% so, at position 3 about 675% so, at position 2 about 200% so, and at position 1 about 350% so. These derivatives have respective sequences:

| | |
|---|---|
| Ac-Val Asp Glu Cys Trp Arg | (AF35910; SEQ ID NO:6) |
| Ile Ile Ala Ser His Ala Trp | |
| Phe Cys Ala Glu Glu-NH₂; | |
| Ac-Val Asp Glu Cys Trp Arg | (AF359 12; SEQ ID NO:8) |
| Ile Ile Ala Ala His Thr Trp | |
| Phe Cys Ala Glu Glu-NH₂; | |
| Ac-Val Asp Ala Cys Trp Arg | (AF35918; SEQ ID NO:13) |
| Ile Ile Ala Ser His Thr Trp | |
| Phe Cys Ala Glu Glu-NH₂; | |
| Ac-Val Ala Glu Cys Trp Arg | (AF35920; SEQ ID NO:14) |
| Ile Ile Ala Ser His Thr Trp | |
| Phe Cys Ala Glu Glu-NH₂; | |
| and | |
| Ac-Ala Asp Glu Cys Trp Arg | (AF35921; SEQ ID NO:15) |
| Ile Ile Ala Ser His Thr Trp | |
| Phe Cys Ala Glu Glu-NH₂. | |

In some embodiments, SEQ ID NO:1 is modified by truncation or addition at one or more of the C and N termini, with substitution or addition at one or both ends of a Lysine (Lys) at the C-terminus or within 3 residues thereof, and substitution of an AHX residue at the N-terminal end or within 3 residues thereof. Resulting preferred species include:

(AF36172; SEQ ID NO:17)

Ac- Glu Cys Trp Arg Ile Ile Ala Ser His Thr Trp
Phe Cys Ala Glu Lys-NH₂;
and

(AF36238; SEQ ID NO:34)

Ac-AHX Val Asp Glu Cys Trp Arg Ile Ile Ala Ser His
Thr Trp Phe Cys Ala Glu Glu-NH₂.

In some embodiments, Lys is substituted for the alanine substitutions of SEQ ID NOs: 6 and 8, in which case residues 1 and 2 are optionally missing, and the N-terminus optionally acylated. Preferred species include:

(AF36552/AF36556; SEQ ID NO:39)

Ac- Glu Cys Trp Arg Ile Ile Ala Ser His Lys Trp
Phe Cys Ala Glu Glu-NH₂;
and

(AF36552/AF36556; SEQ ID NO:41)

Ac- Glu Cys Trp Arg Ile Ile Ala Lys His Thr Trp
Phe Cys Ala Glu Glu-NH₂.

In some embodiments, the species are monomers that may or may not possess an intramolecular disulfide bridge between cysteine residues.

In some embodiments, individual monomers are joined to form dimers, e.g., by disulfide bridges between one or more cysteine residues on the respective monomers, in which case the above limitations on number of residues are addressed only to the monomer. Thus, a dimer would contain even more residues, the exact number depending on whether it is a hetero or homodimer and, if the former, whether or not each monomer thereof has the same number of residues.

In some dimer embodiments, the dimers may alternatively or conjunctively comprise a different type of linker, e.g., one formed from employment of a linker, e.g., a bifunctional linker, e.g., as described in http://www.perbio.com.cn/PIERCE/Technique/crosslink/homocros.pdf. Illustrative examples include the following:

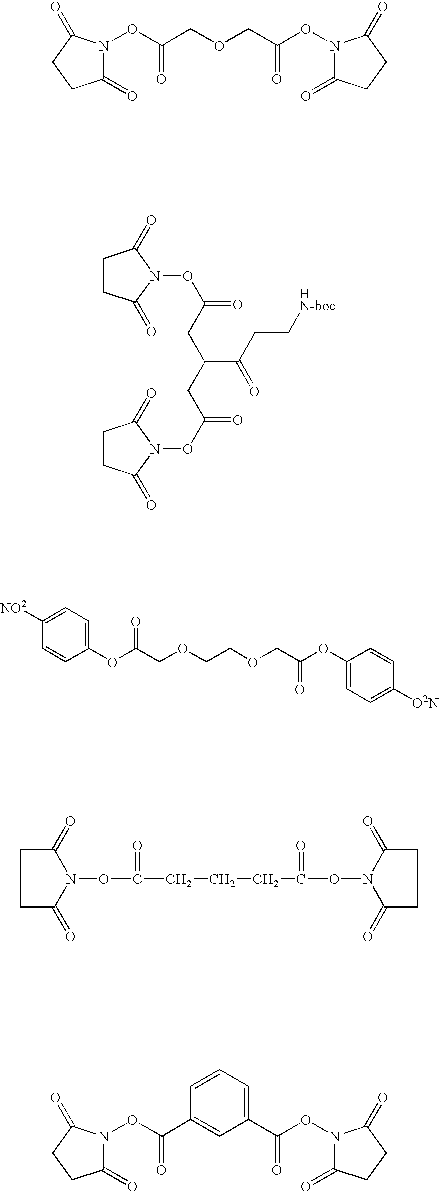

In some embodiments, Glu at position 3 in SEQ ID NO:1 is substituted with another amino acid, preferably one selected from the group consisting of Pro, Asp, D entantiomers thereof, caged glutamic acid (N-[1-(2-Nitrophenyl)ethyloxycarbonyl]glutamic Acid), squarmate (see Chan et al. (1995) J. Med. Chem. 38, 4433; http://www.science.uwaterloo.ca/˜jhonek/UnnaturaLAA.html), or Aib (aminoisobutyric acid). Preferred embodiments of this also incorporate a C-terminal Lys residue, optionally in substitute of Glu in SEQ ID NO: 1 that can be conveniently linked to another Lys at the same position on another optionally identical monomer using one of the above linkers to form a dimer. Preferred species include:

(AF36834/AF36835; SEQ ID NO:49)

Ac- Val Asp Glu Cys Trp Arg Ile Ile Ala Lys His
Thr Trp Phe Cys Ala Glu Lys-NH₂;

(AF36814/AF36819; SEQ ID NO:54)

Ac- Val Asp Pro Cys Trp Arg Ile Ile Ala Lys His
Thr Trp Phe Cys Ala Glu Lys-NH₂;

(AF36844/AF36845; SEQ ID NO:59)

Ac- Val Asp (e) Cys Trp Arg Ile Ile Ala Lys His
Thr Trp Phe Cys Ala Glu Lys-NH₂;
and

(AF36835/AF36839; SEQ ID NO:60)

Ac- Val Asp (Aib) Cys Trp Arg Ile Ile Ala Lys His
Thr Trp Phe Cys Ala Glu Lys-NH₂;

Preferably, the foregoing are dimerized via their N-terminal Lys residues. In the parentheses above, the first AF# is used to denote the monomer and the second AF# is used to denote the dimer.

Other preferred embodiments are monomers joined by their Lys or AHX groups (as the case may be) to form dimers as shown:

| | |
|---|---|
| | (SEQ ID NO: 61) |
| 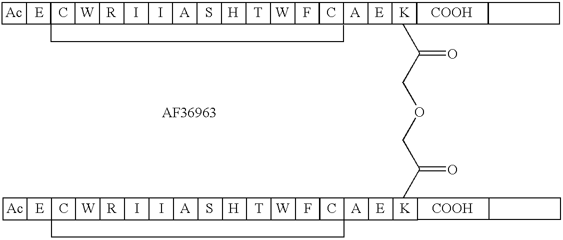 | |
| | (SEQ ID NO: 62) |
| 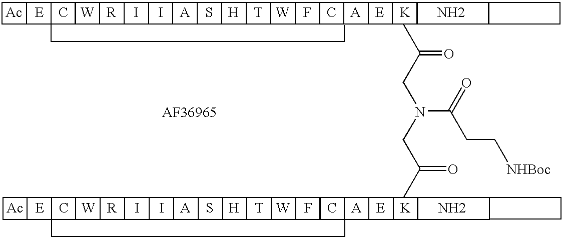 | |
| | (SEQ ID NO: 63) |
| 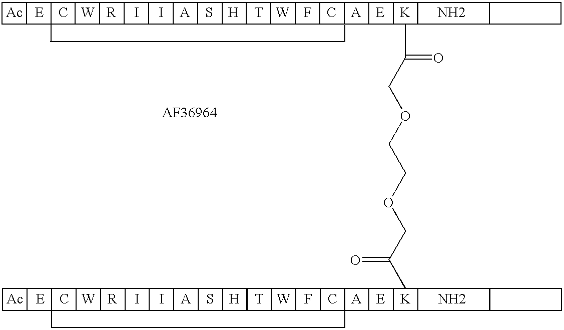 | |
| | (SEQ ID NO: 64) |
| 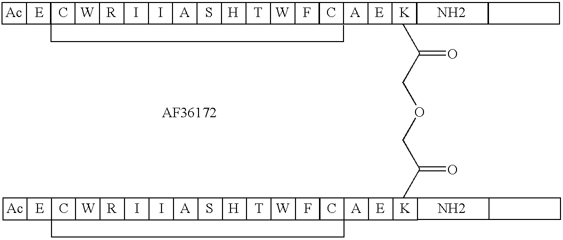 | |
| | (SEQ ID NO: 65) |
| 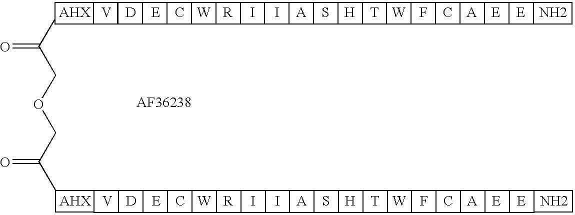 | |
| | (SEQ ID NO: 66) |
| 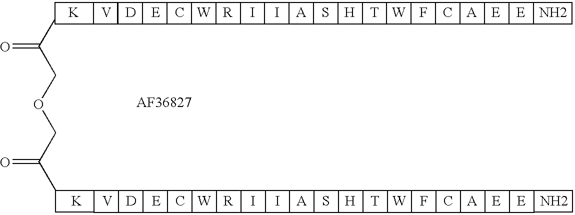 | |
| | (SEQ ID NO: 68) |
| 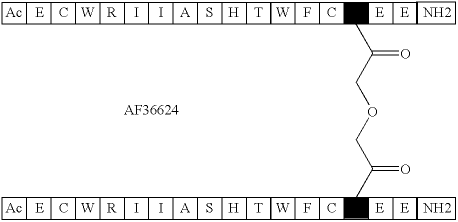 | |
| | (SEQ ID NO: 69) |
| 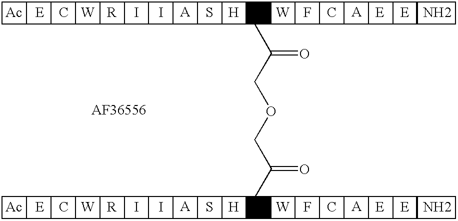 | |
| | (SEQ ID NO: 70) |
| 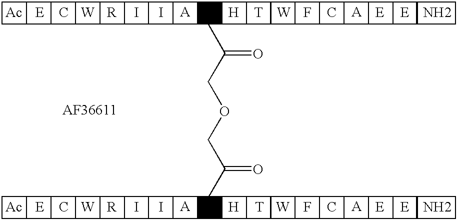 | |
| | (SEQ ID NO: 71) |
| 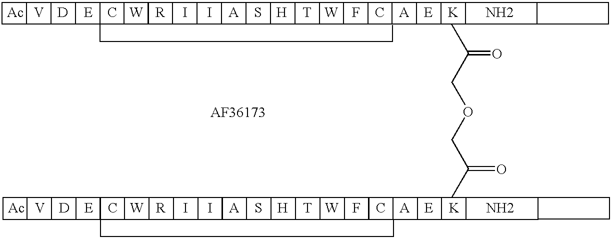 | |
| | (SEQ ID NO: 72) |
| 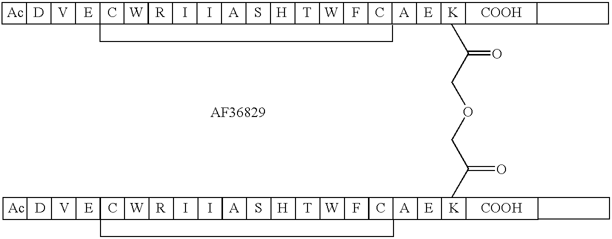 | |

For ease of communication and visualization, the above dimers are shown with the standard one-letter code as opposed to the standard three-letter code. Thus, for example, D is aspartic acid (Asp), V is valine (Val), E is glutamic acid (Glu), C is cysteine (Cys), W is tryptophan (Trp), R is arginine (Arg), I is isoleucine (Ile), A is alanine (Ala), S is serine (Ser), H is histidine (His), T is threonine (Thr), F is phenylalanine (Phe), K is lysine (Lys), AHX is amino hexanoic acid (having structure 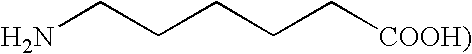
etc., etc. Use of non-standard amino acids and amino acid analogs as known in the art is also contemplated. See, e.g., http://www.emdbiosciences.com/Products/BrowseProductsByCategory.asp?catid=1004; Peptech's Amino Acid Encyclopedia: http://www.peptechcorp.com/documents/PepTech2003_{—}2004.pdf.

Peptide “mimetics” are also envisioned wherein one or more of the peptide linkages [—C(O)NR—] above are substituted with a non-peptidyl linkage such as a —CH₂-carbamate linkage [—CH₂—OC(O)NR—]; a phosphonate linkage; a —CH₂-sulfonamide [—CH₂—S(O)₂NR—] linkage; a urea [—HC(O)NH—] linkage; a —CH₂-secondary amine linkage; or an alkylated peptidyl linkage [—C(O)NR⁶— wherein R⁶ is a lower alkyl]; peptides wherein the N-terminus is derivatized to a —NRR¹ group; to a —NRC(O)R group; to a —NRC(O)OR group; to a —NRS(O)2 R group; to a —NHC(O)NHR group wherein R and R¹ are hydrogen or lower alkyl with the proviso that R and R¹ are not both hydrogen; to a succinimide group; to a benzyloxycarbonyl-NH—(CBZ-NH—) group; or to a benzyloxycarbonyl-NH— group having from 1 to 3 substituents on the phenyl ring selected from the group consisting of lower alkyl, lower alkoxy, chloro, and bromo; or peptides wherein the C terminus is derivatized to —C(O)R² where R² is selected from the group consisting of lower alkoxy, and —NR³R⁴ where R³ and R⁴ are independently selected from the group consisting of hydrogen and lower alkyl.

The compounds of the invention can also be pharmaceutically acceptable acid and base salts of the foregoing peptides where appropriate, which can have the advantage of facilitating dissolution in solution, thereby improving the overall solubility and delivery of the compound to a patient, e.g., in intravenous methods of administration.

Pharmaceutical compositions are also envisioned, as the above compounds will likely find utility in treating patients with one or more afflictions mediated through the interleukin-5 receptor. Such compositions can include one or more of the above peptides or peptide mimetics combined with one or more pharmaceutically acceptable excipients, e.g., stabilizers, bulking agents, salts, surfactants, etc. These pharmaceutical compositions can be in a variety of forms including oral dosage forms, as well as inhalable powders and solutions and injectable and infusible solutions.

In another aspect, the invention features methods of using the above peptides and peptide mimetics for therapeutic purposes in treating disorders mediated by IL-5 or involving improper production of or response to IL-5. The compounds of the invention can be used to inhibit production and accumulation of eosinophils. These compounds will find particular use in the treatment of asthma. Thus, in some aspects and embodiments, the present invention provides a method for treating a patient having a disorder that is susceptible to treatment with an IL-5 inhibitor. The patient is administered a therapeutically effective dose or amount of a compound of the present invention. Combinational therapies are also envisioned, e.g., using one or more beta-adrenergic agonists such as albuterol, terbutaline, formoterol, fenoterol, and prenaline, and/or one or more anti-inflammatory corticosteroids such beclomethasome, triamcinolone, flurisolide, and dexamethasone, and/or ipratropium bromide in combination with the above compounds. In addition, other antihistamines, e.g., Claritin®, can be administered, as can anti-IL9 receptor antibodies or antagonists.

In another aspect, the invention features use of the above peptides and peptide mimetics in diagnostic methods. Thus, diagnostic kits and methods are also contemplated. In these embodiments, the peptides and peptide mimetics can be labeled with a detectable label. Accordingly, the peptides and peptide mimetics without such a label can serve as intermediates in the preparation of labeled peptides and peptide mimetics.

Other features, uses, and advantages of the invention will be apparent to the person of ordinary skill in the art from the drawings, detailed description of the invention, and claims to follow.

### DESCRIPTION OF THE DRAWINGS

FIG. 1 shows the results of an alanine scan of 2-cys monomer AF17121 (SEQ ID NO: 1) as measured using an AlphaQuest™ binding assay.

FIG. 2 shows optimization of various C- and N-terminal dimers as measured using each of AlphaQuest™ binding, TF-1 and Eosinophil assays. Shown are both C- and N-terminal truncations, substitutions of N- and C-terminal residues with Lys and/or AHX, and dimerization of such compounds using these residues.

FIG. 3 shows mid-chain lysine scan AlphaQuest™ binding assay data for various embodiments of the invention.

FIG. 4 shows AlphaQuest™ and TF-1 assay optimization by substituting different amino acids and amino acid analogs for the N-terminal glutamic acid (Glu) residue in AF36834 (monomer) and AF36835 (dimer using DIG linker).

FIG. 5 shows TF-1 assay results for various C-terminal-linked dimers.

FIG. 6 shows additional N- and C-terminal dimer assay data (Eosinophil or AlphaQuest™).

FIG. 7 shows internal-linked dimer AlphaQuest™ binding assay data.

FIG. 8 shows C-terminal dimer AlphaQuest™ binding assay data.

### DETAILED DESCRIPTION OF THE PREFERRED EMBODIMENTS

I. Overview

The present invention provides compounds that bind to the IL-5 R. These compounds include “lead” peptide compounds and “derivative” compounds constructed so as to have the same or similar molecular structure or shape as the lead compounds but that differ from the lead compounds either with respect to susceptibility to hydrolysis or proteolysis and/or with respect to other biological properties, such as increased affinity for the receptor. The present invention also provides compositions comprising an effective IL-5R binding, IL-5 blocking compound, and more particularly a compound, that is useful for treating disorders associated with the overexpression of IL-5 or with the production and accumulation of eosinophils.

II. Definitions

The following definitions are set forth to illustrate and define the meaning and scope of the various terms used to describe the invention herein.

“Pharmaceutically acceptable salts” refer to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid. Representative salts include the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napsylate, and the like.

“Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, menthanesulfonic acid, ethanesulfonic acid, p-toluenesulfonic acid, salicylic acid and the like. For a description of pharmaceutically acceptable acid addition salts as prodrugs, see Bundgaard, H., supra.

“Pharmaceutically acceptable ester” refers to those esters which retain, upon hydrolysis of the ester bond, the biological effectiveness and properties of the carboxylic acid or alcohol and are not biologically or otherwise undesirable. For a description of pharmaceutically acceptable esters as prodrugs, see Bundgaard, H., ed., (1985) Design of Prodrugs, Elsevier Science Publishers, Amsterdam. These esters are typically formed from the corresponding carboxylic acid and an alcohol. Generally, ester formation can be accomplished via conventional synthetic techniques. (See, e.g., March Advanced Organic Chemistry, 3rd Ed., John Wiley & Sons, New York (1985) p. 1157 and references cited therein, and Mark et al. Encyclopedia of Chemical Technology, John Wiley & Sons, New York (1980).) The alcohol component of the ester will generally comprise (i) a C2-C12 aliphatic alcohol that can or can not contain one or more double bonds and can or can not contain branched carbon chains or (ii) a C7-C12 aromatic or heteroaromatic alcohols. This invention also contemplates the use of those compositions which are both esters as described herein and at the same time are the pharmaceutically acceptable acid addition salts thereof.

“Pharmaceutically acceptable amide” refers to those amides which retain, upon hydrolysis of the amide bond, the biological effectiveness and properties of the carboxylic acid or amine and are not biologically or otherwise undesirable. For a description of pharmaceutically acceptable amides as prodrugs, see Bundgaard, H., ed., (1985) Design of Prodrugs, Elsevier Science Publishers, Amsterdam. These amides are typically formed from the corresponding carboxylic acid and an amine. Generally, amide formation can be accomplished via conventional synthetic techniques. (See, e.g., March Advanced Organic Chemistry, 3rd Ed., John Wiley & Sons, New York (1985) p. 1152 and Mark et al. Encyclopedia of Chemical Technology, John Wiley & Sons, New York (1980).) This invention also contemplates the use of those compositions which are both amides as described herein and at the same time are the pharmaceutically acceptable acid addition salts thereof.

The claim terms “variation,” “variant,” “replaced,” “substitute,” “substituted,” “substitution,” “deacylated,” and “deamidated” do not necessarily connote a chemical modification of SEQ ID NO: 1 as substrate. Rather, these terms are primarily used to distinguish SEQ ID NO:1. Thus, the compounds of the invention may be generated either by starting with SEQ ID NO:1 and chemically modifying it, or alternatively by engaging in de novo synthesis, whether synthetically and/or recombinantly produced, and whether with SEQ ID NO:1 in mind or not. In addition, and consistent with these definitions, the terms “deacylated” and “non-acylated” can be synonymous, as can the terms “deamidated” and “non-amidated.”

“Pharmaceutically or therapeutically acceptable carrier” refers to a carrier medium which does not interfere with the effectiveness of the biological activity of the active ingredients and which is not toxic to the host or patient.

“Stereoisomer” refers to a chemical compound having the same molecular weight, chemical composition, and constitution as another, but with the atoms grouped differently. That is, certain identical chemical moieties are at different orientations in space and, therefore, when pure, has the ability to rotate the plane of polarized light. However, some pure stereoisomers may have an optical rotation that is so slight that it is undetectable with present instrumentation. The compounds of the instant invention may have one or more asymmetrical carbon atoms and therefore include various stereoisomers. All stereoisomers are included within the scope of the invention.

“Therapeutically- or pharmaceutically-effective amount” as applied to the compositions of the instant invention relents to the amount of composition sufficient to induce a desired biological result. That result can be alleviation of the signs, symptoms, or causes of a disease, or any other desired alteration of a biological system. In the present invention, the result will typically involve a decrease in the immunological and/or inflammatory responses to infection or tissue injury.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G. The foregoing assumes “L” designations as known in the art. Small case single letters denote the corresponding “D” enantiomer. For example, “e” corresponds to the D-enantiomer of “E” (Glutamic Acid), etc., etc.

In addition to peptides consisting only of naturally-occurring amino acids, peptidomimetics or peptide analogs are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂ NH—, —CH₂ S—, —CH₂—CH₂—, —CH.dbd.CH—(cis and trans), —COCH₂—, —CH(OH)CH₂—, and —CH₂ SO—, by methods known in the art and further described in the following references: Spatola, A. F. in CHEMISTRY AND BIOCHEMISTRY OF AMINO ACIDS, PEPTIDES, AND PROTEINS, B. Weinstein, eds., Marcel Dekker, New York, p. 267 (1983); Spatola, A. F., Vega Data (March 1983), Vol. 1, Issue 3, PEPTIDE BACKBONE MODIFICATIONS (general review); Morley, Trends Pharm Sci (1980) pp. 463-468 (general review); Hudson, D. et al., (1979) Int J Pept Prot Res 14:177-185 (—CH2 NH—, CH2 CH2—); Spatola et al., (1986) Life Sci 38:1243-1249 (—CH2—S); Hann (1982) J. Chem. Soc. Perkin Trans. I 307-314 (—CH—CH—, cis and trans); Almquist et al., (1980) J Med Chem 23:1392-1398 (—COCH₂—); Jennings-White et al., (1982) Tetrahedron Lett 23:2533 (—COCH₂—); Szelke et al., (1982) European Appin. EP 45665 CA: 97:39405 (1982) (—CH(OH)CH₂—); Holladay et al., (1983) Tetrahedron Lett 24:4401-4404 (—C(OH)CH₂—); and Hruby (1982) Life Sci 31:189-199 (—CH₂—S—); each of which is incorporated herein by reference. A particularly preferred non-peptide linkage is —CH₂ NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) (e.g., immunoglobulin superfamily molecules) to which the peptidomimetic binds to produce the therapeutic effect. Derivitization (e.g., labeling) of peptidomimetics should not substantially interfere with the desired biological or pharmacological activity of the peptidomimetic. Generally, peptidomimetics of receptor-binding peptides bind to the receptor with high affinity and possess detectable biological activity (i.e., are agonistic or antagonistic to one or more receptor-mediated phenotypic changes).

Systematic substitution of one or more amino acids of a consensus sequence with a D-amino acid of the same type (e.g., D-lysine in place of L-lysine) may be used to generate more stable peptides. Unless specifically indicated by the use of a “small case,” e.g., in the case of Glutamic Acid an “e” as opposed to an “E”, the instance of the amino acids in the compounds of the invention are predominantly, if not entirely, of the “L” variety as understood in the art, although the term “variant” may connote either “L” or “D.” In addition, constrained peptides comprising a consensus sequence or a substantially identical consensus sequence variation may be generated by methods known in the art (Rizo and Gierasch (1992) Ann. Rev. Biochem. 61: 387, incorporated herein by reference); for example, by adding internal cysteine residues capable of forming intramolecular disulfide bridges which cyclize the peptide.

Combinational therapies are also envisioned whereby activity of the IL5 antagonists of the invention are supplemented by the addition of one or more other pharmacologically active compounds that enhance or add to their overall ameliorative or preventative effect. Examples include the use of additional antihistamines such as Clariting and/or anti-IL9 receptor antibodies or antagonists. Anti-inflammatory agents that may be administered with the compounds of the invention include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, stilindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

III. Binding Affinity to the IL-5 Receptor

Binding affinity to IL-5R can be evaluated using various binding assays, facilitated in that the IL-5 receptor and its extracellular alpha and beta chains have been cloned and are immobilizable using various mediums that allow for ligand binding affinity assessment.

The immobilized alpha. chain, beta. chain, and heterodimer, as well as the extracellular domains of the single chains of the IL-5 receptors were produced in recombinant host cells. The DNA encoding IL-5R was obtained by PCR of cDNA from TF-1 cells using primers obtained from the published receptor sequences. See Murata (1992) J. Exp. Med. 175:341-351 and Hayashida (1990) Proc. Natl. Acad. Sci. USA 87:9655-9659, each of which is incorporated herein by reference. One useful form of IL-5R is constructed by expressing the protein as a soluble protein in baculovirus transformed host cells using standard methods; another useful form is constructed with a signal peptide for protein secretion and for glycophospholipid membrane anchor attachment. This form of anchor attachment is called “PIG-tailing”. See Caras and Wendell (1989) Science 243:1196-1198 and Lin et al. (1990) Science 249:677-679.

Using the PIG-tailing system, one can cleave the receptor from the surface of the cells expressing the receptor (e.g., transformed CHO cells selected for high level expression of receptor with a cell sorter) with phospholipase C. The cleaved receptor still comprises a carboxy terminal sequence of amino acids, called the “HPAP tail”, from the signal protein for membrane attachment and can be immobilized without further purification. The recombinant receptor protein can be immobilized by coating the wells of microtiter plates with an anti-HPAP tail antibody (Ab 179), blocking non-specific binding with bovine serum albumin (BSA) in PBS, and then binding cleaved recombinant receptor to the antibody. Using this procedure, one should perform the immobilization reaction in varying concentrations of receptor to determine the optimum amount for a given preparation, because different preparations of recombinant protein often contain different amounts of the desired protein. In addition, one should ensure that the immobilizing antibody is completely blocked (with IL-5R or some other blocking compound) during the affinity enrichment process. Otherwise, unblocked antibody can bind undesired phage during the affinity enrichment procedure. One can use peptides that bind to the immobilizing antibody to block unbound sites that remain after receptor immobilization to avoid this problem or one can simply immobilize the receptor directly to the wells of microtiter plates, without the aid of an immobilizing antibody. See U.S. patent application Ser. No. 07/947,339, filed Sep. 18, 1992, incorporated herein by reference.

To discriminate among higher affinity peptides, a monovalent receptor probe frequently is used. This probe can be produced using protein kinase A to phosphorylate a kemptide sequence fused to the C-terminus of the soluble receptor. The “engineered” form of the IL-5 receptor alpha and beta chains are then expressed in host cells, typically CHO cells. Following PI-PLC harvest of the receptors, the receptor is labeled to high specific activity with ³³P for use as a monovalent probe to identify high affinity ligands.

### EXAMPLE 1

### Solid Phase Peptide Synthesis

Various peptides of the invention were synthesized using the Merrifield solid phase synthesis techniques (see Steward and Young, Solid Phase Peptide Synthesis, 2nd. Edition, Pierce Chemical, Rockford, Ill. (1984), and Merrifield, J. Am. Chem. Soc. 1963, 85, 2149) on an Applied Biosystems Inc. Model 431A or 433A peptide synthesizer. Alternatively, a Symphony multiple channel peptide synthesizer could be used.

The peptides were assembled using standard protocols of the Applied Biosystems Inc. System Software version 1.01. Each coupling was performed for one hour with HBTU and HOBt. Double-couplings were performed at each step.

The resin used was TentaGel R RAM resin from RAPP Polymere GmbH or NovaSyn TGA resin preloaded with an N-alpha-Fmoc protected amino acids from Novabiochem with a capacity of 0.18 mmol/g. Use of TentaGel R RAM resin results in a carboxyl terminal amide functionality upon cleavage of the peptide from the resin. Upon cleavage, the preloaded NovaSyn TGA resin produces a carboxylic acid moiety at the C-terminus of the final product. Most reagents, resins, and protected amino acids (free or on the resin) were purchased from Novabiochem or Applied Biosystems Inc.

Trityl (Trt) and/or acetamidomethyl (Acm) were utilized as protecting groups for Cys residues. The Fmoc group was used for amino protection during the coupling procedure. Primary amine protection on amino acids was achieved with Fmoc and side chain protection groups were t-butyl for serine, tyrosine, aspartic acid, glutamic acid, and threonine; Pbf (2,2,4,6,7-pentamethyldihydrobenzofuran-5-sulfonyl) or Pmc (2,2,5,7,8-pentamethylchroman-6-sulfonyl) for arginine; N-t-butyloxycarbonyl for tryptophan and lysine; and N-trityl for histidine, asparagines, and glutamine. Side chain protecting groups are not shown in the figures for convenience.

After the desired amino acid sequence has been completed, the desired peptide is decoupled from the resin support by treatment with a cleavage reagent, such as reagent K (82.5% trifluoroacetic acid, 5% water, 5% phenol, 5% thioanisole, 2.5% 1,2-ethanedithiol) or slight modifications of this reagent, which not only cleaves the peptide from the resin, but also cleaves all remaining side chain protecting groups. The deprotected peptides were precipitated with diethyl ether.

The crude peptides were purified by preparative, reverse-phase, high performance liquid chromatography on a C18 bonded silica gel column with a gradient of acetonenitrile/water containing 0. 1% trifluoroacetic acid. The homogeneous peptides were characterized by HPLC, LC/MS or MALDI/MS, and amino acid analysis when applicable.

### EXAMPLE 2

### Formation of Disulfide Bonds in 2-Cys Family Peptides

The crude peptide obtained directly from a cleavage/deprotection procedure as described above was dissolved and vigorously stirred in a DMSO/water (50/50) solution with a concentration of 1 mg peptide/mL. The reaction progress was monitored by HPLC (by the shift of retention time) and LC/MS (by the change of molecular weight). After the reaction was complete, the crude product was concentrated under lyophilization, and further purified by preparative HPLC as mentioned above.

### EXAMPLE 3

### Dimerization of 2-Cys Family Peptides via Lysine Residues

Peptides with a free primary amino group, such as the amino group on the lysine side chain or the amino group of aminocaproic acid residue, can be dimerized by reacting with a bifunctional linker. The dimerization can be achieved by treatment of the oxidized and purified peptide with 0.5 molar equivalent of a bifunctional linker in DMF (N,N-dimethylformamide) or DMSO (methylsulfoxide) in the presence of an excess amount of triethylamine or diisopropylethylamine. The reaction was monitored by HPLC (by the shift of retention time) and LC/MS (by the change of molecular weight). After the reaction was complete, the reaction mixture was directly loaded onto preparative HPLC for purification. Below are some examples of bifunctional linkers used for peptide dimerization:

### EXAMPLE 4

### Synthesis of AF36172: Representative Procedure

Preparation of monomer AF36157: 350 mg TentaGel R RAM resin from RAPP Polymere GmbH, with a capacity of 0.18 mmol/g, was loaded on the Symphony™ multiple synthesizer. The peptide was assembled using standard protocols of the Protein Technologies Inc. System Software version Win 2.01. Each coupling was performed for 45 min with DIC and HOBt. Double-couplings were performed at each step. After the sequence was completed, the peptide was cleaved from the resin support by treatment with reagent K (82.5% trifluoroacetic acid, 5% water, 5% phenol, 5% thioanisole, 2.5% 1,2-ethanedithiol) for 2 hours, which not only cleaved the peptide from the resin, but also cleaved all remaining side chain protecting groups. The deprotected peptide was precipitated with diethyl ether. The crude peptide was then dissolved in DMSO/water (50:50) in a concentration of 1 mg peptide/mL and vigorously stirred for 2 days at room temperature for oxidation. The reaction mixture was concentrated under lyophilization, and purified by preparative HPLC to yield 53 mg of pure monomer AF36157 as a white powder.

### EXAMPLE 5

### Dimerization of AF36157 with a DIG Linker

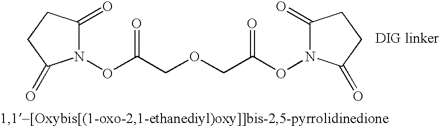

Monomer AF36157 (53 mg) and DIG linker (4 mg) were dissolved in anhydrous DMF (4 mL). To this mixture was added a few drops of triethyl amine. The reaction mixture was agitated at room temperature for a few hours. The reaction was monitored by HPLC (by the shift of retention time) and LC/MS (by the change of molecular weight). After the reaction was complete, the reaction mixture was directly loaded onto preparative HPLC for purification. The desired fractions were collected and lyophilized to give 30 mg of dimer AF36172 as a white powder.

Following synthesis and purification of the different monomeric and dimeric peptides as described above, the peptides were subjected to the following assays that allow for a determination of binding affinity to the IL-5R and an assessment of potential use in an in vivo system.

### EXAMPLE 6

### IL-5 Receptor-Ligand Alpha Quest Binding Assay Using Biotinylated-MBP-AF12368

The affinity of compounds for the IL-5 receptor was tested in a homogeneous Alpha Quest binding assay. The peptides tested were solubilized at approximately 10 mM in DMSO. Each peptide was then serially diluted into freshly made, sterile filtered assay buffer (40 mM Hepes, pH 7.4 with NaOH, containing 1 mM MgCl₂, 0.1% BSA and 0.05% Tween-20). Four microliters of each test compound dilution was transferred in triplicate to a 384-well, low volume, white, polypropylene Griener plate (E&K Scientific #31075). A mixture was prepared in assay buffer containing Packard Bioscience AlphaScreen streptavidin coated donor beads at 40 ug/uL, MAb179 (prepared as described in Whitehom, E., A Generic Method for Expression and Use of “Tagged” Soluble Versions of Cell Surface Receptor, Biotechnolgy 1995, 13, 1215-1219) conjugated Packard Bioscience Alpha Screen acceptor beads per manufactures protocol, at 40 ug/mL, and human IL-5Rα extracellular domain harvest with a MAb 179 epitope tag prepared as previously described (England, B., A Potent Dimeric Peptide Antagonist of Interleukin-5 that Binds Two Interleukin-5 Receptor α Chains, PNAS, vol.97, no. 12, 6862-6867). Two microliters of beads and receptor harvest mixture were added to each assay well. Alpha Quest beads are light sensitive so all steps after the introduction of the beads were carried out under green lighting (lights adapted with Roscolux Chroma Green #389 filter sleeves which transmit <1% at 680 nm). After a 5-10 minute room temperature incubation, two microliters of 62.4 nM biotinylated MBP-AF12368, GEVCTRDVANHRWMCGVD (SEQ ID NO 73), tracer peptide (prepared as described in Schatz P., Use of Peptide Libraries to Map the Substrate Specificity of a Peptide-Modifying Enzyme: A 13 Residue Consensus Peptide Specifies Biotinylation in Escherichia coli, Biotechnology 1993, 11, 1138-1143) diluted in assay buffer was added to each well. The assay plate was spun to 55 rpm in a plate centrifuge, sealed with Packard Top Seal, wrapped in foil and placed in a room temperature incubator in the instrument room. The plate was read after 16-20 hours using a Packard Bioscience Alpha Quest Discovery™ plate reader.

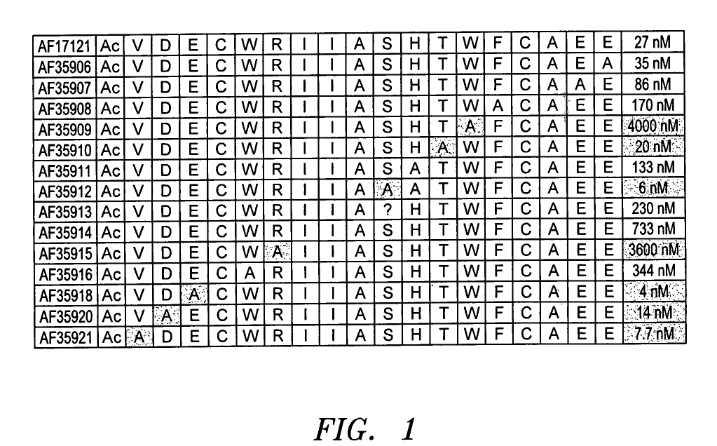

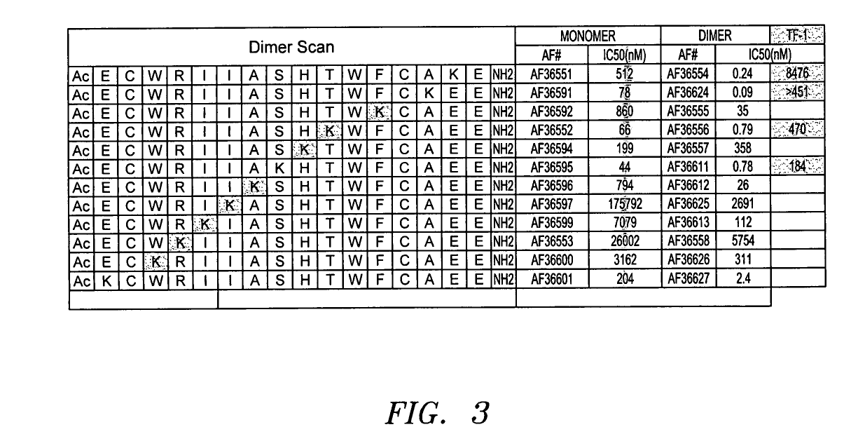

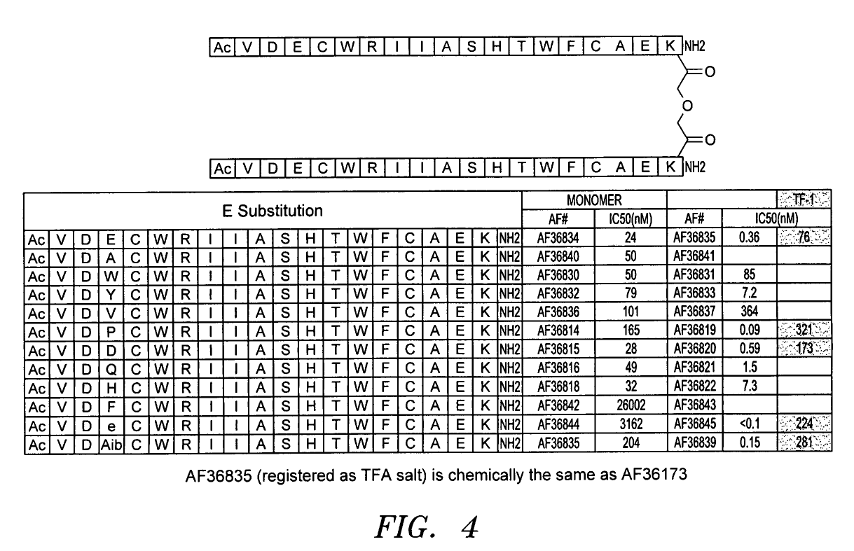

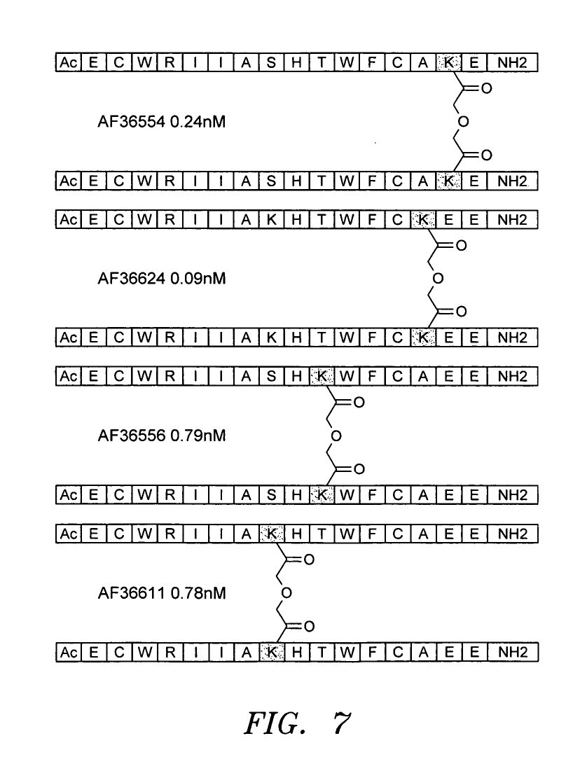

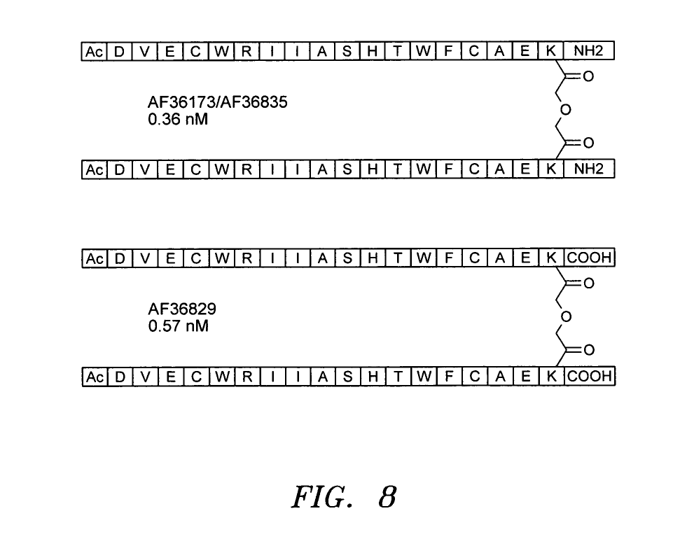

Results for various compound embodiments of the invention are depicted in FIGS. 1-4 and 6-8.

### EXAMPLE 7

### TF-1 Proliferation Assay

Materials used in conjunction with this example included a Human Bone Marrow Erythroblast TF-1 Cell Line (from premyeloid erythroleukemia patient, DNAX, IL-5 dependent), RPMI 1640 media (Mediatech Cellgro, Cat No. 15-040-CV), Fetal Bovine Serum (FBS, Hyclone, Cat No. SH30071.03), L-glutarnine (HyClone, Cat No. SH30034.01), Recombinant Human IL-5 (R&D system, Cat No. 205-IL-005), Recombinant Human GM-CSF (R&D systems, Cat No. 215-GM-005), TF-1 Growth Medium (RPMI 1640, 10% FBS, 2mM L-glutamine, 2ng/ml rhGM-CSF), Assay Medium (RPMI 1640, 10% FBS, 2mM L-glutamine), MTS and PMS (Promega, Cat No. G5430), Microtest™ tissue culture treated plate, 96 well (Becton Dickinson, Cat No. 353072), and Polypropylene 96well plates (Agilent Technologies, Part No. 5042-1385)

Bone Marrow Erythroblast TF-1 cells were grown as a suspension in a flask or spinner, depending on need, at 37° C., 5% CO2, with the cell population doubling in about 30 hours. Cells were split to 1×10ˆ5 cells/ml for use in 3-4 days. 1:3 serial dilutions of peptide, IL-5 or GMCSF were prepared under sterile conditions in 96 well polypropylene plates to establish a curve, with 12 points taken for each curve including the medium control which contained only medium. If the peptide was insoluble in the medium, it should not be filtered. The peptide and positive control (AF18748) were diluted to 4× final desired starting concentration, with the final DMSO concentration in the well with cells not exceeding 1%. Cells were washed 3× with assay medium and adjusted to a concentration of 10ˆ5/ml, after which 50 ul of the washed cells were added to each well of the above 96 well culture plates. 50 ul/well of IL-5 or GMCSF, or 25 ul /well of peptide dilution were transferred in duplicates from the dilution plate to a culture plate containing 5000 cells/well. The culture plate was pre-incubated for 30 min at 37° C., 5% CO₂, in a humidified incubator, after which 25 ul/well of IL-5 (1 ng/ml final concentration) was added to each of the wells to establish a peptide dilution curve. The culture plate was then incubated for 70 to 72 hours at 37° C., 5% CO2, in a humidified incubator, and checked daily with a microscope. Following this incubation, frozen MTS and PMS solutions were thawed at room temperature or at 37° C. in a water bath, a 1:20 mix of PMS:MTS then quickly established, 20 ul/well quickly added to each well of the culture plate, and the plate then allowed to incubate 1-4 hours at 37° C., 5% CO2, in a humidified incubator. The plate was then subjected to an ELISA plate reader with absorbance recorded at 490 nm for each well, and a nonlinear regression curve fit program was then used to calculate the IC₅₀ of each peptide.

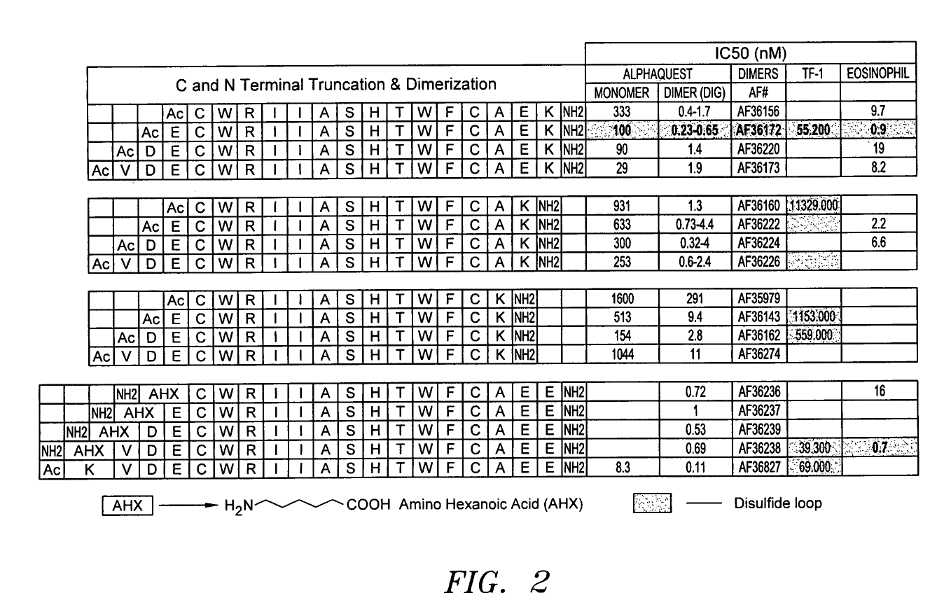

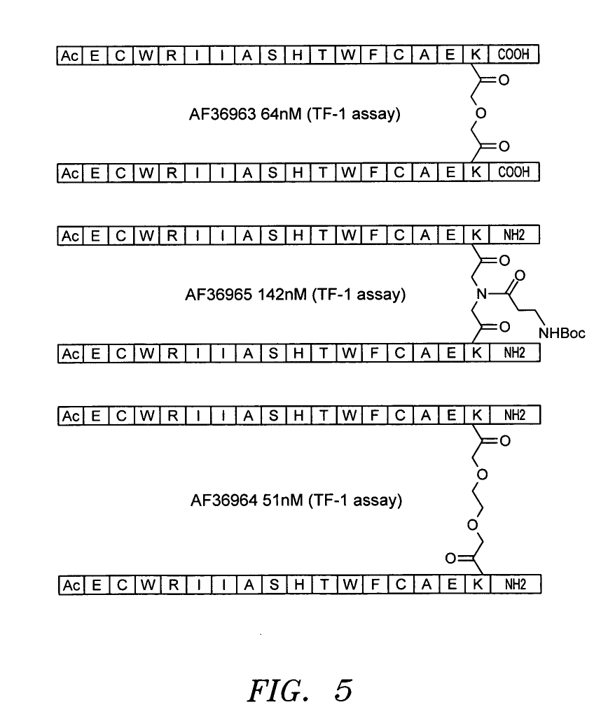

TF-1 assay results are depicted in FIGS. 2-5 for various compound embodiments of the invention.

### EXAMPLE 8

### Eosinophil Attachment Assay

Material used in connection with this example included Whole Blood Buffy Coat (Stanford Medical School Blood Center, from 1 unit of blood), Human Eosinophil Enrichment Column Kit (R&D systems, Cat No. HEEC-B), Recombinant Human IL-5 (R&D system, Cat No. 205-IL-005), Mouse anti-Human CD-16 (CATAG, Cat No. MHCD1600-4), Dynabeads M-450 Sheep anti-Mouse IgG (Dynal Biotech, Prod No. 110.02), Human IgG (Sigma, I-2511), Phosphate Buffer Saline without calcium and magnesium (Gibco, Cat No. 12388-013), Tween-20 (Calbiochem, Cat No. 655205), Human Albumin Solution (HAS, 5%, Bayer, Cat No. 82-327-1), Hank's Balanced Salt Solution (Invitrogen Life Technology, Cat No. 14065-056), Sodium Citrate (trisodium salt, Sigma, Cat No. S-4641), Fetal Bovine Serum (FBS, Hyclone, Cat No. SH30071.03),PBST (PBS+0.05% Tween-20), PBS++ (PBS+0.1% HAS+0.38% Trisodium citrate), HBSS+(Assay medium, HBSS+0.1% HAS), Polypropylene 96well plate (Agilent Technologies, Part No. 5042-1385), Microtiter plate (Nunc-Immuno Maxisorp Plates, VWR Cat No. 62409-024), Dynal MPC-1 (Dynal, Cat No. 120-01), Dynall MPC-S (Dynal, Cat No. 120.20), May-Grunwald Stain (Sigma, Cat No. MG-500), Harleco Phosphate Buffer, ph 6.8 (EM Science, Cat No. 1218), CTAB (Cetyltrimethylammonium Bromide, CALBIOCHEM, Cat No. 219374), and Sigma Fast o-PD Tablet Sets (Sigma, Cat No. P-9187).

Buffy coat cells (20-30 mls) were diluted 1:1 with PBS (without calcium and magnesium), an equal volume of RBC Gradient Solution (R&D system, Cat No. 205-IL-005) mixed in gently, and the resulting mixtures let stand at room temperature for 30-45min. to allow RBC sedimentation. White blood cells (WBCs) were then loaded onto a density gradient to fractionate mononuclear cells from PMNs. PMN Enrichment was performed by placing 20 ml of PMN separation medium (R&D, Cat No. HEEC-B) into each of 3 fifty ml polypropylene centrifuge tubes. The top plasma layer of cells was carefully collected from the RBC Gradient tubes, pooled, ⅓ of the pool carefully overlayed onto each of the 3 tubes containing the PMN separation medium, the overlayed tubes centrifuged at 500 g for 30 min at room temperature, the resulting supernatant discarded along with any cells that may have attached to the sides of the tube, and the cell pellet containing the PMNs retained. The PMN cell pellet was then resuspended in 2-3ml of sterile PBS. Residual red blood cells (RBCs) were then lysed by adding 24 ml of sterile water to the cells in 2-3 ml of sterile PBS, gently inverting, and then quickly adding 8 ml of a 3.5% solution of filter-sterilized NaCl solution (w/v) to make the solution isotonic. The cells were then spun down gently, washed with lx PBS, resuspend in 2 ml of PBS++, the cell count measured using a hemocytometer, and the cell density then adjusted to 10ˆ7/ml with PBS++. CD-16 negative selection was then accomplished using mouse anti-CD16 antibody mixed with the cells at a ratio of 2 ug of anti-CD16 per 10ˆ7 cells, with the mixture then incubated on ice or in cold room for 20 min with rotation and tilting. During the incubation, enough dynalbeads for 2-4 dynalbeads/cell were added to a new 15 ml tube and the beads washed with PBS++ twice by placing the washing tube in a magnet (Dynal MPC-1) for 1 minute, pipetting off the fluid, and resuspending the beads with 2 ml of PBS++. The incubated cells were then washed 2× with PBS++ to get rid of the free anti-CD16, and the cells resuspended to 10ˆ7/ml concentration. The bead suspension was then added to the cells, the mixture incubated on ice for 30 min with tilting or rotation, the beads removed with a magnet, and the resulting fluid saved, spun and the resulting cells resuspend with 1 ml of HBSS+. Residual beads were removed with a magnet (MPC-S), the cells washed twice with assay medium (HBSS+), re-suspended in 1 ml of assay medium, and the cell number and viability checked using Trypan blue and a May-Grunwald Staining procedure. Briefly, cells were smeared on glass slides, the slides air-dryed, placed in methanol for 1 min, followed by May-Grunwald Stain solution for 4 min. and stain-buffer mixture (10 ml of May-Grunwald Stain, 15 ml of pH 6.4 to 6.8 buffer, 35 ml of Deionized water) for 8 min. The stain-buffer mixture was then drained, the slides rinsed with 10-15 ml of DI water, left to air dry at room temp., and examined microscopically for the purity of the eosinophils. An eosinophil attachment assay was then conducted as follows: Peptide and IL-5 dilutions were made in 96 well polypropylene plates under totally sterile conditions, with 12 points taken for each curve including the medium control. The peptides were then diluted with assay medium to 4× final desired starting concentration, with the final DMSO concentration in the wells with cells not exceeding 1%. Serial 1:3 dilutions were then made from well #2 to well # 11, leaving the last well as medium only. Microtiter plates were coated with human IgG at lug/well in 100 ul of PBS for 1-2 hours at room temperature with shaking, the plates then washed 2× with PBST, blocked with 200 ul/well of 50% FBS (FCS) in PBS for 1-2 hours at room temperature with shaking, washed again with PBST 2×, then HBSS+2×, and 50 ul of 10ˆ5/ ml then added to each well of the coated plates. 25 ul/well of peptide dilutions were then added in duplicates from the dilution plate to the coated plate containing cells, and 25 ul/well of assay medium to the IL-5 control rows. The plate was then pre-incubated for 5 min at 37° C., 5% CO2, in a humidified incubator, after which 25 ul/well of IL-5 (150 ng/ml) was added to the rows (controls included) to establish a peptide dilution curve, the plate then incubated on ice for 10 min to allow the cells to settle, followed by incubation of the plate for 30 min at 37° C., 5% CO₂, in a humidified incubator. The cells were then lysed by the addition of 50 ul/well of 0.3% CTAB to lyse the cells, 100 ul/well of OPD substrate added, the plate developed at room temperature for 15 to 30 min, 50 ul/well of 2N sulfuric acid then added, and the absorbance recorded at 490nm using an ELISA plate reader. The data is then fit using a 4 parameter nonlinear regression curve fitting program to generate an IC₅₀ value for each peptide.

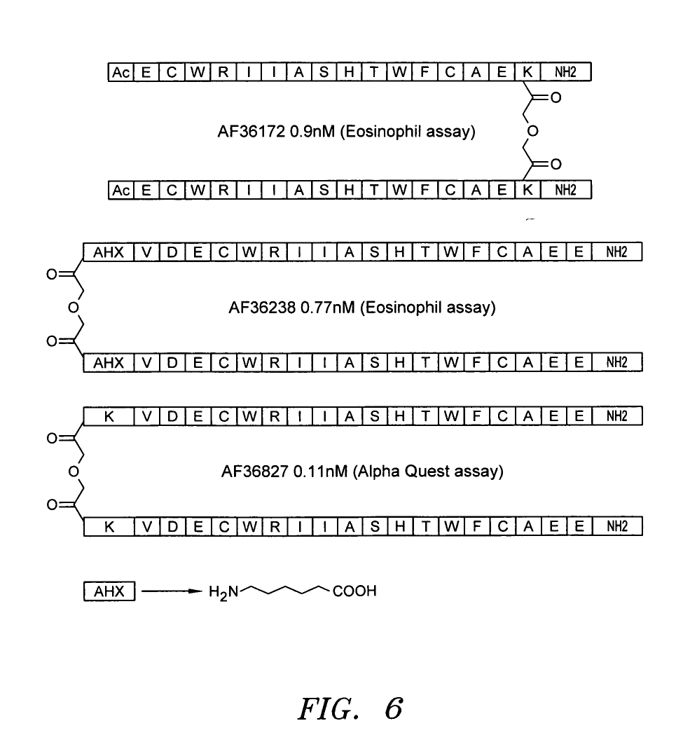

Eosinophil assay results are depicted in FIGS. 2 and 6 for various compound embodiments of the invention.

One of ordinary skill will appreciate that the parameters described in the above can be adjusted depending on the conditions used, and depending on whether and to what extent the methods of formulation and amounts of materials used are scaled up or down, or varied, with respect to one another.

The foregoing examples are not limiting and merely representative of various aspects and embodiments of the present invention. All documents and web-page pages cited are indicative of the levels of skill in the art to which the invention pertains. The disclosure of each document and web-page cited is incorporated by reference herein to the same extent as if each had been incorporated by reference in its entirety, although none of the documents is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention as defined by the scope of the claims.

The reagents described herein are either commercially available or else readily producible without undue experimentation using routine procedures known to those of ordinary skill in the art. Illustrative vendors include Sigma-Aldrich (St. Louis, Mo., U.S.A.) and Pierce Biotechnology (Rockford, Ill., U.S.A.).

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms, and each has a different meaning within the patent laws. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

Other embodiments are within the following claims.

1. A compound comprising a peptide sequence 12 to 40 amino acid residues in length, or pharmaceutically acceptable salt or ester thereof, said peptide sequence or pharmaceutically acceptable salt or ester thereof comprising a variation of sequence Ac-Val Asp Glu Cys Trp Arg Ile Ile Ala Ser His Thr Trp Phe Cys Ala Glu Glu-NH2 (SEQ ID NO:1) wherein said variation comprises one or more of:
an alanine substitution at one or more of residues 1, 2, 3, 10, and 13;
a core sequence comprising residues 3-17 of SEQ ID NO: 1 and having a lysine substitution at one or more of residues 3, 10, 12, 15, and 16;
a substitution, truncation or addition to within 3 residues of the C- or N-terminus and having a C- or N-terminal lysine or AHX residue;
a core sequence comprising residues 3-17 of SEQ ID NO: 1, a C-terminal lysine residue substituted for residue 17 and a proline, aspartic acid, or D form of glutamic acid (e) substituted for residue 3;
the C-terminus is deamidated;
the N-terminus is deacylated;
an intramolecular cysteine-cysteine disulfide bridge;
an intermolecular cysteine-cysteine disulfide bridge that is present as part of a dimer; and
a Lys-Lys or AHX-AHX bridge is present as part of a dimer.

2. The compound of claim 1 that comprises an intramolecular disulfide bridge and that is selected from the following group of compounds:

| | |
|---|---|
| | SEQ ID NO: 2 |
| Ac-Val Asp Glu Cys Trp Arg Ile Ile Ala Ser His Ala Trp Phe Cys Ala Glu Glu-NH₂; | |
| (AF35910;) | |
| | SEQ ID NO: 3 |
| Ac-Val Asp Glu Cys Trp Arg Ile Ile Ala Ala His Thr Trp Phe Cys Ala Glu Glu-NH₂: | |
| (AF35912;) | |
| | SEQ ID NO: 4 |
| Ac-Val Asp Ala Cys Trp Arg Ile Ile Ala Ser His Thr Trp Phe Cys Ala Glu Glu-NH₂; | |
| (AF35918;) | |
| | SEQ ID NO: 5 |
| Ac-Val Ala Glu Cys Trp Arg Ile Ile Ala Ser His Thr Trp Phe Cys Ala Glu Glu-NH₂; | |
| (AF35920;) | |
| | SEQ ID NO: 6 |
| Ac-Ala Asp Glu Cys Trp Arg Ile Ile Ala Ser His Thr Trp Phe Cys Ala Glu Glu-NH₂; | |
| (AF35921;) | |
| | SEQ ID NO: 7 |
| Ac-Glu Cys Trp Arg Ile Ile Ala Ser His Thr Trp Phe Cys Ala Glu Lys-NH₂; | |
| (AF36172;) | |
| | SEQ ID NO: 8 |
| Ac-AHX Val Asp Glu Cys Trp Arg Ile Ile Ala Ser His Thr Trp Phe Cys Ala Glu Glu-NH₂; | |
| (AF36238;) | |
| | SEQ ID NO: 9 |
| Ac-Glu Cys Trp Arg Ile Ile Ala Ser His Lys Trp Phe Cys Ala Glu Glu-NH₂; | |
| (AF36552/AF36556;) | |
| | SEQ ID NO: 10 |
| Ac-Glu Cys Trp Arg Ile Ile Ala Lys His Thr Trp Phe Cys Ala Glu Glu-NH₂; | |
| (AF36552/AF36556;) | |
| | SEQ ID NO: 11 |
| Ac-Val Asp Glu Cys Trp Arg Ile Ile Ala Lys His Thr Trp Phe Cys Ala Glu Lys-NH₂; | |
| (AF36834/AF36835;) | |
| | SEQ ID NO: 12 |
| Ac-Val Asp Pro Cys Trp Arg Ile Ile Ala Lys His Thr Trp Phe Cys Ala Glu Lys-NH₂; | |
| (AF36814/AF36819;) | |
| | SEQ ID NO: 13 |
| Ac-Val Asp (e) Cys Trp Arg Ile Ile Ala Lys His Thr Trp Phe Cys Ala Glu Lys-NH₂; | |
| (AF36844/AF36845;) | |
| | SEQ ID NO: 14 |
| Ac-Val Asp (Aib) Cys Trp Arg Ile Ile Ala Lys His Thr Trp Phe Cys Ala Glu Lys-NH₂; | |
| (AF36835/AF36839;) | |
| | (SEQ ID NO: 15) |
| 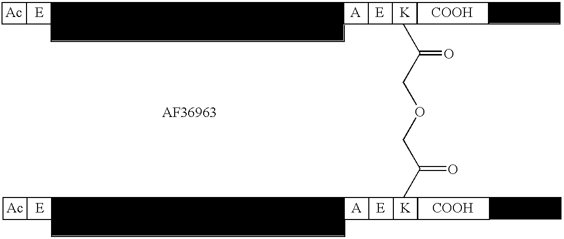 | |
| | (SEQ ID NO: 16) |
| 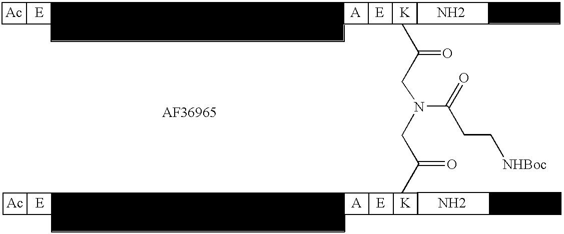 | |
| | (SEQ ID NO: 17) |
| 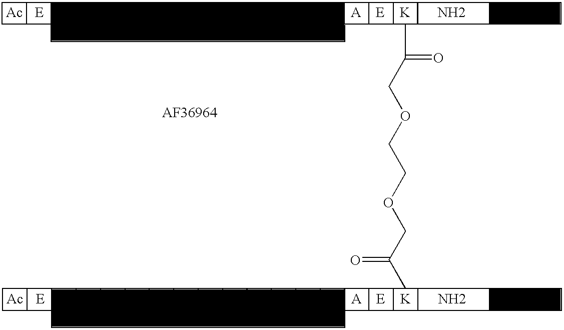 | |
| | (SEQ ID NO: 18) |
| 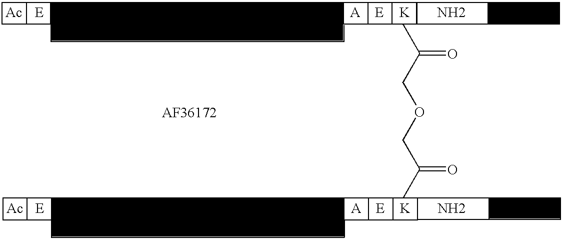 | |
| | (SEQ ID NO: 8) |
| 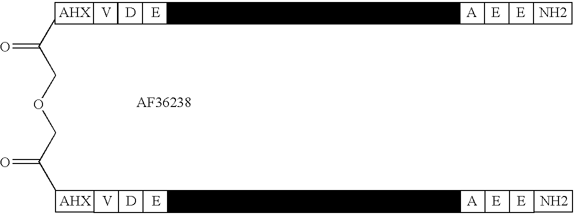 | |
| | (SEQ ID NO: 19) |
| 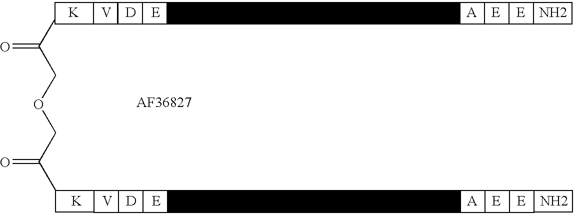 | |
| | (SEQ ID NOS: 20 and 21) |
| 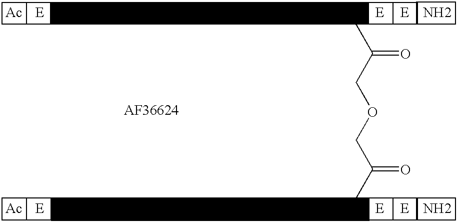 | |
| | (SEQ ID NO: 22) |
| 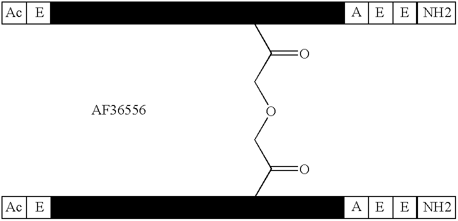 | |
| | (SEQ ID NO: 23) |
| 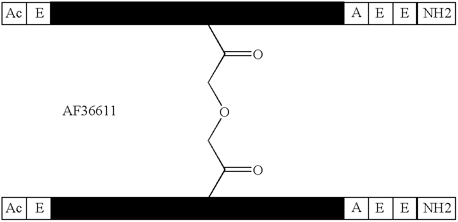 | |
| | (SEQ ID NO: 24) |
| 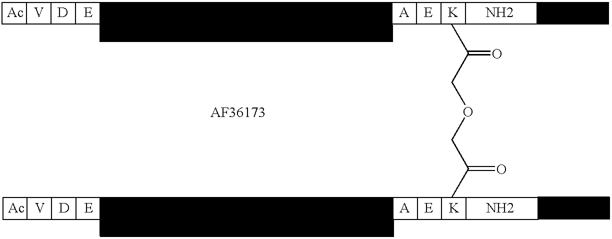 | |
| | (SEQ ID NO: 25) |
| 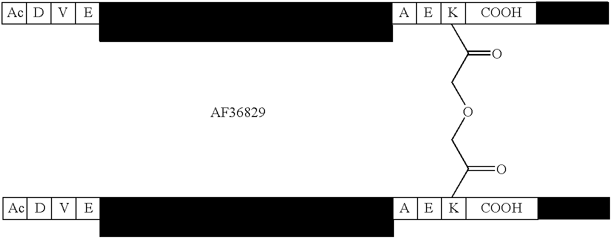 | |

and pharmaceutically acceptable salts and esters of the foregoing group, non-acylated versions of the foregoing group, and non-amidated versions of the foregoing group.

3. The compound of claim 2 wherein said compound is a monomer.

4. The compound of claim 2 wherein said compound is a dimer.

5. The compound of claim 4 wherein said dimer is a homodimer.

6. The compound of claim 4 wherein said dimer is fashioned using a bifunctional linker, optionally one selected from the group consisting of:

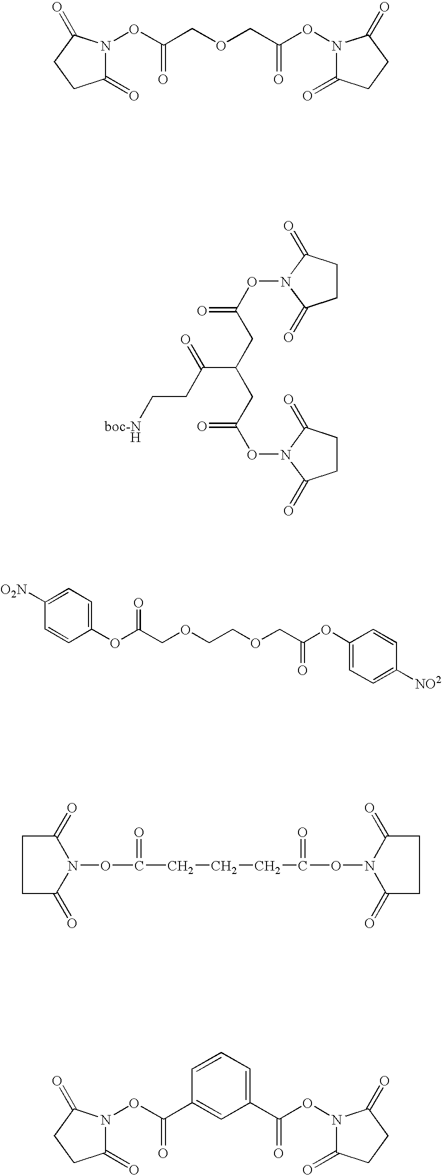

7. A pharmaceutical composition comprising one or more compounds or pharmaceutically acceptable salts according to any one of claims 1-6, further comprising one or more pharmaceutically acceptable excipients, optionally further comprising one or more antihistamines, and optionally further still comprising one or more anti-IL9 receptor antibodies or antagonists.

8. A method of ameliorating or preventing an IL-5 receptor mediated disorder comprising administering to a patient in need thereof a pharmaceutically effective amount of one or more compounds according to any one of claims 1-6; and optionally further comprising administering one or more members selected from the group of antihistamines and anti-IL9 receptor antibodies or antagonists.

9. The method of claim 8 wherein said compound is accompanied by one or more pharmaceutically acceptable excipients.

10. The method of claim 8 wherein said affliction is asthma.

11. The method of claim 8 wherein said patient is human.

12. The method of claim 8 wherein said one or more compounds is administered in tandem or sequentially with a beta-adrenergic agonist compound.

13. The method of claim 12 wherein said beta-adrenergic agonist compound is selected from the group consisting of albuterol, terbutaline, formoterol, fenoterol, and prenaline.

14. The method of claim 8 or 12 wherein said compound is administered in conjunction with an anti-inflammatory corticosteroid.

15. The method of claim 14 wherein said anti-inflammatory corticosteroid is selected from the group consisting of beclomethasome, triamcinolone, flurisolide, and dexamethasone.

16. The method of claim 8 wherein the compound is administered in conjunction with ipratropium bromide.


SEQUENCE LISTING (70 sequences)

SEQ ID NO: 1 - protein, 18 aa, Artificial Sequence
  Description of Artificial Sequence Synthetic peptide
VDECWRIIASHTWFCAEE

SEQ ID NO: 2 - protein, 18 aa, Artificial Sequence
  Description of Artificial Sequence Synthetic peptide
VDECWRIIASHAWFCAEE

SEQ ID NO: 3 - protein, 18 aa, Artificial Sequence
  Description of Artificial Sequence Synthetic peptide
VDECWRIIAAHTWFCAEE

SEQ ID NO: 4 - protein, 18 aa, Artificial Sequence
  Description of Artificial Sequence Synthetic peptide
VDACWRIIASHTWFCAEE

SEQ ID NO: 5 - protein, 18 aa, Artificial Sequence
  Description of Artificial Sequence Synthetic peptide
VAECWRIIASHTWFCAEE

SEQ ID NO: 6 - protein, 18 aa, Artificial Sequence
  Description of Artificial Sequence Synthetic peptide
ADECWRIIASHTWFCAEE

SEQ ID NO: 7 - protein, 16 aa, Artificial Sequence
  Description of Artificial Sequence Synthetic peptide
ECWRIIASHTWFCAEK

SEQ ID NO: 8 - protein, 19 aa, Artificial Sequence
  Description of Artificial Sequence Synthetic peptide
XVDECWRIIASHTWFCAEE

SEQ ID NO: 9 - protein, 16 aa, Artificial Sequence
  Description of Artificial Sequence Synthetic peptide
ECWRIIASHKWFCAEE

SEQ ID NO: 10 - protein, 16 aa, Artificial Sequence
  Description of Artificial Sequence Synthetic peptide
ECWRIIAKHTWFCAEE

SEQ ID NO: 11 - protein, 18 aa, Artificial Sequence
  Description of Artificial Sequence Synthetic peptide
VDECWRIIAKHTWFCAEK

SEQ ID NO: 12 - protein, 18 aa, Artificial Sequence
  Description of Artificial Sequence Synthetic peptide
VDPCWRIIAKHTWFCAEK

SEQ ID NO: 13 - protein, 18 aa, Artificial Sequence
  Description of Artificial Sequence Synthetic peptide
VDXCWRIIAKHTWFCAEK

SEQ ID NO: 14 - protein, 18 aa, Artificial Sequence
  Description of Artificial Sequence Synthetic peptide
VDXCWRIIAKHTWFCAEK

SEQ ID NO: 15 - protein, 16 aa, Artificial Sequence
  Description of Artificial Sequence Synthetic peptide
ECWRIIASHTWFCAEK

SEQ ID NO: 16 - protein, 16 aa, Artificial Sequence
  Description of Artificial Sequence Synthetic peptide
ECWRIIASHTWFCAEK

SEQ ID NO: 17 - protein, 16 aa, Artificial Sequence
  Description of Artificial Sequence Synthetic peptide
ECWRIIASHTWFCAEK

SEQ ID NO: 18 - protein, 16 aa, Artificial Sequence
  Description of Artificial Sequence Synthetic peptide
ECWRIIASHTWFCAEK

SEQ ID NO: 19 - protein, 19 aa, Artificial Sequence
  Description of Artificial Sequence Synthetic peptide
KVDECWRIIASHTWFCAEE

SEQ ID NO: 20 - protein, 16 aa, Artificial Sequence
  Description of Artificial Sequence Synthetic peptide
ECWRIIASHTWFCKEE

SEQ ID NO: 21 - protein, 16 aa, Artificial Sequence
  Description of Artificial Sequence Synthetic peptide
ECWRIIAKHTWFCKEE

SEQ ID NO: 22 - protein, 16 aa, Artificial Sequence
  Description of Artificial Sequence Synthetic peptide
ECWRIIASHKWFCAEE

SEQ ID NO: 23 - protein, 16 aa, Artificial Sequence
  Description of Artificial Sequence Synthetic peptide
ECWRIIAKHTWFCAEE

SEQ ID NO: 24 - protein, 18 aa, Artificial Sequence
  Description of Artificial Sequence Synthetic peptide
VDECWRIIASHTWFCAEK

SEQ ID NO: 25 - protein, 18 aa, Artificial Sequence
  Description of Artificial Sequence Synthetic peptide
DVECWRIIASHTWFCAEK

SEQ ID NO: 26 - protein, 18 aa, Artificial Sequence
  Description of Artificial Sequence Synthetic peptide
GEVCTRDVANHRWMCGVD

SEQ ID NO: 27 - protein, 18 aa, Artificial Sequence
  Description of Artificial Sequence Synthetic peptide
VDECWRIIASHTWFCAEA

SEQ ID NO: 28 - protein, 18 aa, Artificial Sequence
  Description of Artificial Sequence Synthetic peptide
VDECWRIIASHTWFCAAE

SEQ ID NO: 29 - protein, 18 aa, Artificial Sequence
  Description of Artificial Sequence Synthetic peptide
VDECWRIIASHTWACAEE

SEQ ID NO: 30 - protein, 18 aa, Artificial Sequence
  Description of Artificial Sequence Synthetic peptide
VDECWRIIASHTAFCAEE

SEQ ID NO: 31 - protein, 18 aa, Artificial Sequence
  Description of Artificial Sequence Synthetic peptide
VDECWRIIASATWFCAEE

SEQ ID NO: 32 - protein, 18 aa, Artificial Sequence
  Description of Artificial Sequence Synthetic peptide
VDECWRIAASHTWFCAEE

SEQ ID NO: 33 - protein, 18 aa, Artificial Sequence
  Description of Artificial Sequence Synthetic peptide
VDECWRAIASHTWFCAEE

SEQ ID NO: 34 - protein, 18 aa, Artificial Sequence
  Description of Artificial Sequence Synthetic peptide
VDECWAIIASHTWFCAEE

SEQ ID NO: 35 - protein, 18 aa, Artificial Sequence
  Description of Artificial Sequence Synthetic peptide
VDECARIIASHTWFCAEE

SEQ ID NO: 36 - protein, 15 aa, Artificial Sequence
  Description of Artificial Sequence Synthetic peptide
CWRIIASHTWFCAEK

SEQ ID NO: 37 - protein, 17 aa, Artificial Sequence
  Description of Artificial Sequence Synthetic peptide
DECWRIIASHTWFCAEK

SEQ ID NO: 38 - protein, 14 aa, Artificial Sequence
  Description of Artificial Sequence Synthetic peptide
CWRIIASHTWFCAK

SEQ ID NO: 39 - protein, 15 aa, Artificial Sequence
  Description of Artificial Sequence Synthetic peptide
ECWRIIASHTWFCAK

SEQ ID NO: 40 - protein, 16 aa, Artificial Sequence
  Description of Artificial Sequence Synthetic peptide
DECWRIIASHTWFCAK

SEQ ID NO: 41 - protein, 17 aa, Artificial Sequence
  Description of Artificial Sequence Synthetic peptide
VDECWRIIASHTWFCAK

SEQ ID NO: 42 - protein, 13 aa, Artificial Sequence
  Description of Artificial Sequence Synthetic peptide
CWRIIASHTWFCK

SEQ ID NO: 43 - protein, 14 aa, Artificial Sequence
  Description of Artificial Sequence Synthetic peptide
ECWRIIASHTWFCK

SEQ ID NO: 44 - protein, 15 aa, Artificial Sequence
  Description of Artificial Sequence Synthetic peptide
DECWRIIASHTWFCK

SEQ ID NO: 45 - protein, 16 aa, Artificial Sequence
  Description of Artificial Sequence Synthetic peptide
VDECWRIIASHTWFCK

SEQ ID NO: 46 - protein, 16 aa, Artificial Sequence
  Description of Artificial Sequence Synthetic peptide
XCWRIIASHTWFCAEE

SEQ ID NO: 47 - protein, 17 aa, Artificial Sequence
  Description of Artificial Sequence Synthetic peptide
XECWRIIASHTWFCAEE

SEQ ID NO: 48 - protein, 18 aa, Artificial Sequence
  Description of Artificial Sequence Synthetic peptide
XDECWRIIASHTWFCAEE

SEQ ID NO: 49 - protein, 16 aa, Artificial Sequence
  Description of Artificial Sequence Synthetic peptide
ECWRIIASHTWFCAKE

SEQ ID NO: 50 - protein, 16 aa, Artificial Sequence
  Description of Artificial Sequence Synthetic peptide
ECWRIIASHTWKCAEE

SEQ ID NO: 51 - protein, 16 aa, Artificial Sequence
  Description of Artificial Sequence Synthetic peptide
ECWRIIASKTWFCAEE

SEQ ID NO: 52 - protein, 16 aa, Artificial Sequence
  Description of Artificial Sequence Synthetic peptide
ECWRIIAKHTWFCAEE

SEQ ID NO: 53 - protein, 16 aa, Artificial Sequence
  Description of Artificial Sequence Synthetic peptide
ECWRIIKSHTWFCAEE

SEQ ID NO: 54 - protein, 16 aa, Artificial Sequence
  Description of Artificial Sequence Synthetic peptide
ECWRIKASHTWFCAEE

SEQ ID NO: 55 - protein, 16 aa, Artificial Sequence
  Description of Artificial Sequence Synthetic peptide
ECWRKIASHTWFCAEE

SEQ ID NO: 56 - protein, 16 aa, Artificial Sequence
  Description of Artificial Sequence Synthetic peptide
ECWKIIASHTWFCAEE

SEQ ID NO: 57 - protein, 16 aa, Artificial Sequence
  Description of Artificial Sequence Synthetic peptide
ECKRIIASHTWFCAEE

SEQ ID NO: 58 - protein, 16 aa, Artificial Sequence
  Description of Artificial Sequence Synthetic peptide
KCWRIIASHTWFCAEE

SEQ ID NO: 59 - protein, 18 aa, Artificial Sequence
  Description of Artificial Sequence Synthetic peptide
VDACWRIIASHTWFCAEK

SEQ ID NO: 60 - protein, 18 aa, Artificial Sequence
  Description of Artificial Sequence Synthetic peptide
VDWCWRIIASHTWFCAEK

SEQ ID NO: 61 - protein, 18 aa, Artificial Sequence
  Description of Artificial Sequence Synthetic peptide
VDYCWRIIASHTWFCAEK

SEQ ID NO: 62 - protein, 18 aa, Artificial Sequence
  Description of Artificial Sequence Synthetic peptide
VDVCWRIIASHTWFCAEK

SEQ ID NO: 63 - protein, 18 aa, Artificial Sequence
  Description of Artificial Sequence Synthetic peptide
VDPCWRIIASHTWFCAEK

SEQ ID NO: 64 - protein, 18 aa, Artificial Sequence
  Description of Artificial Sequence Synthetic peptide
VDDCWRIIASHTWFCAEK

SEQ ID NO: 65 - protein, 18 aa, Artificial Sequence
  Description of Artificial Sequence Synthetic peptide
VDQCWRIIASHTWFCAEK

SEQ ID NO: 66 - protein, 18 aa, Artificial Sequence
  Description of Artificial Sequence Synthetic peptide
VDHCWRIIASHTWFCAEK

SEQ ID NO: 67 - protein, 18 aa, Artificial Sequence
  Description of Artificial Sequence Synthetic peptide
VDFCWRIIASHTWFCAEK

SEQ ID NO: 68 - protein, 18 aa, Artificial Sequence
  Description of Artificial Sequence Synthetic peptide
VDXCWRIIASHTWFCAEK

SEQ ID NO: 69 - protein, 18 aa, Artificial Sequence
  Description of Artificial Sequence Synthetic peptide
VDXCWRIIASHTWFCAEK

SEQ ID NO: 70 - protein, 18 aa, Artificial Sequence
  Description of Artificial Sequence Synthetic peptide
DVECWRIIASHTWFCAEK